(19) United States
(12) Patent Application Publication
(10) Pub. No.: US 2013/0105593 A1
(43) Pub. Date: May 2, 2013

(54) AIR FRESHENER

(71) Applicant: Henry Wu, New Taipei City (TW)

(72) Inventor: Henry Wu, New Taipei City (TW)

(73) Assignee: Aromate Industries Co., Ltd., New Taipei City (TW)

(21) Appl. No.: 13/281,508

(22) Filed: Oct. 26, 2011

(51) Int. Cl. A61L 9/04; B65B 3/02

(52) U.S. Cl. 239/34; 53/452

(57) ABSTRACT

An air freshener of the invention comprises a carrier body having a recessed portion, a resilient clip portion extending from one end of the carrier body to mount the air freshener on an air vent, a head arranged on the end of the carrier body and a scent portion received in the recessed portion for diffusing fragrance. The scent portion can be formed by pouring and solidifying scent gel in the recessed portion of the carrier body.

### BACKGROUND OF THE INVENTION

1. Field of the Invention

The invention relates to an air freshener, especially to an air freshener that is compact in structure and easy to manufacture.

2. Description of the Related Art

There are a variety of air fresheners that can be placed in front of an automobile's cool air vent to improve a car's interior air quality. With reference to FIG. 7, a conventional air freshener for a vehicle comprises a head 50, a clip 52 coupled to the head 50 extending perpendicularly outward from the head 50, and sized and shaped to be inserted into a vehicle air vent and clipped to a vehicle air vent cover such that the head 50 is adjacent the vehicle air vent, and including a pair of vent rods 52a, 52b sized and shaped to be inserted into the vehicle air vent and having a plurality of protrusions 53, a pair of coherent elastomeric polymer bodies 54a, 54b having a plurality of apertures 55, each of the bodies 54a, 54b coupled to a different one of the vent rods 52a, 52b via coupling of the plurality of apertures 55 with the plurality of protrusions 53, and insertable into the vehicle air vent with the vent rods 52a, 52b and a scent material, interspersed within the bodies 54a, 54b, capable of diffusing out of the bodies 54a, 54b to surrounding air. However, the conventional air freshener is complicated in structure and is not easy to manufacture.

Therefore, it is necessary to provide an air freshener which is compact in structure and easy to manufacture.

### SUMMARY OF THE INVENTION

It is an object of this invention to provide an air freshener which is compact in structure and easy to manufacture.

In order to achieve the above objects, the air freshener according to an embodiment of the present invention comprises a carrier body having a recessed portion along its periphery, a resilient clip portion extending from the carrier body, a head arranged on one end of the carrier body and a scent portion receivable in the recessed portion, the scent portion being at least partially in the form of an elastic band such that the scent portion is at least partially held in the recessed portion by the elasticity of the scent portion.

The present invention further provides an air freshener comprising a carrier body having a recessed portion along its periphery, the recessed portion having at least one slot, a resilient clip portion extending from the carrier body, a head arranged on one end of the carrier body and a scent gel poured and solidified in the recessed portion and the at least one slot to form a scent portion that interlocks with the carrier body.

The present invention further provides an air freshener comprising a carrier body having a recessed portion thereon, a resilient clip portion extending from the carrier body, a head arranged on one end of the carrier body and a scent gel poured and solidified in the recessed portion.

The present invention further provides a method of making an air freshener comprising the steps of: forming a carrier body with a recessed portion along its periphery, at least one slot in the recessed portion and a resilient clip portion extending from one end of the carrier body; arranging a head on the end of the carrier body; pouring scent gel to fill the at least one slot and the recessed portion; and solidifying the scent gel to form a scent portion. The step of arranging the head can be coincidental with the step of pouring scent gel and includes pouring scent gel in a mold to integrally form the head with the scent portion when the scent gel is solidified.

The present invention further provides a method of making an air freshener comprising the steps of: forming a carrier body with a recessed portion thereon and a resilient clip portion extending from one end of the carrier body; arranging a head on the end of the carrier body; pouring scent gel to fill the recessed portion; and solidifying the scent gel to form a scent portion.

In an embodiment of the present invention, the head is inclined relative to the carrier body.

In an embodiment of the present invention, the carrier body has a pivotal hole on the end and the head has a pivotal shaft inserted into the pivotal hole such that the head pivots with respect to the carrier body.

In an embodiment of the present invention, the pivotal shaft is engaged with the pivotal hole by the frictional force therebetween such that the head can pivot and hold its position at any pivotal angle of the head relative to the carrier body.

In an embodiment of the present invention, the outer surface of the pivotal shaft and the inner surface of the pivotal hole respectively forms engaging ribs to enhance the engagement between the pivotal shaft and the pivotal hole.

In an embodiment of the present invention, the head is made of scent material and is integrally formed with the scent portion.

In an embodiment of the present invention, the head is formed in any of the following shapes: circle, rectangle, square, doll-like shape, heart shaped and irregular shape.

### DESCRIPTION OF THE DRAWINGS

FIG. 1 illustrates an exploded view of an air freshener according to an alternative embodiment of the present invention;

FIG. 2 illustrates a schematic view showing the scent portion engaging the carrier body;

FIG. 3 illustrates a schematic view of the scent portion of the air freshener with the head in a heart-like shaped according to an alternative embodiment of the present invention.

FIG. 4 illustrates a schematic view of the carrier body of the air freshener with swivelable head according to another alternative embodiment of the present invention;

FIG. 5 illustrates a cross-section view showing the air freshener of the present invention placed in a (gel filling) mold; and

FIGS. 6a and 6b are top and side views of an air freshener according to another alternative embodiment of the present invention.

FIGS. 7a and 7b illustrate a conventional air freshener.

### DETAILED DESCRIPTION OF THE INVENTION

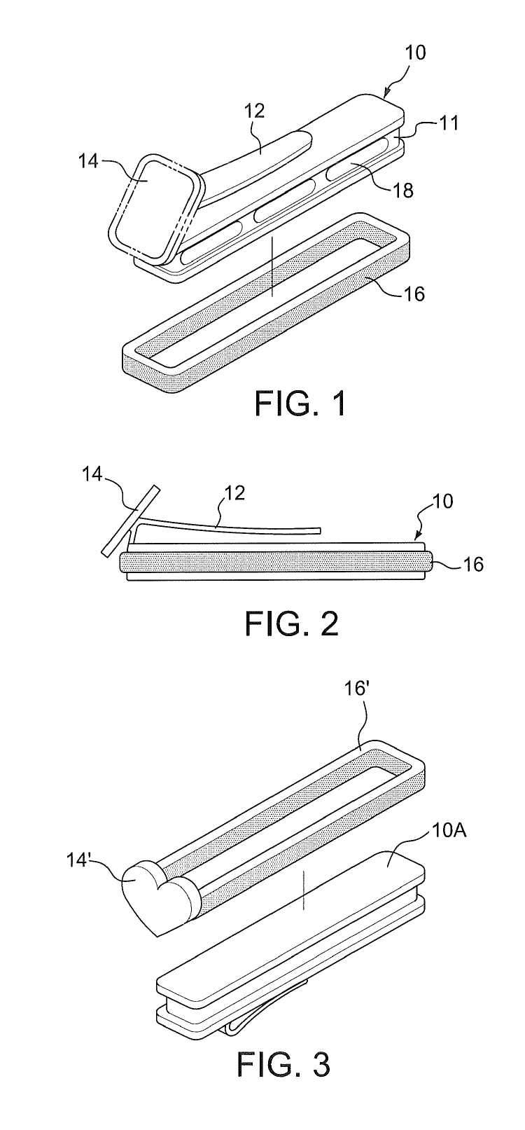

As shown in FIGS. 1 and 2, the air freshener of a preferred embodiment of the invention includes a carrier body 10 having a recessed portion 11 along its periphery, a resilient clip portion 12 extending from one end of the carrier body 10, a head 14 and a scent portion 16. The air freshener of the invention can be inserted into and fixed to a louver or fin of the article, e.g. an air vent of an air conditioner, by the resilient clip portion 12. In the embodiment shown in FIGS. 1 and 2, the head 14 is formed on the resilient clip portion 12. Alternatively, the head 14 can be formed directly on the carrier body 10. In addition, the head 14 is inclined relative to the carrier body 10 as shown in FIGS. 1 and 2 or perpendicular to the carrier body 10. Further, the head 14 can be of any shape to meet the user's demand, such as circular, rectangular, square, heart shaped, doll-like shaped, irregular shape and so on; patterns or words for decoration or promotion can be imprinted or printed on the head.

The scent portion 16 contains scent material for providing fragrance. The scent material is perfume oil or impregnated with perfume oil. The scent material may disperse or diffuse out of the material of the scent portion, which is a polymer, into the air or atmosphere to be discernable or smelled. The scent portion 16 is in the form of a band and can be at least slightly stretched because of the elasticity of the material. The band-like scent portion 16 is capable of being received in the recessed portion 11 of the carrier body 10. In assembling the carrier body 10 and the scent portion 16, the scent portion 16 is first hooked on one end of recessed portion 11 of the carrier body 10; then the scent portion 16 is stretched so as to pass over the lower surface of the carrier body 10 and finally the scent portion 16 is released and its shape is restored due to its elasticity such that the scent portion 16 is received and held in the recessed portion 11 of the carrier body 10.

In another preferred embodiment of the invention as shown in FIG. 3, the head 14′ is not formed on the clip portion 12 or the carrier body 10A, but integrally formed with the scent portion 16′. As indicated above, the head 14′ can be of any shape and is heart shaped in accordance with the embodiment shown in FIG. 3. The method of assembling the scent portion 16′ to the carrier body is the same as the embodiment shown in FIGS. 1 and 2 and thus is omitted for brevity.

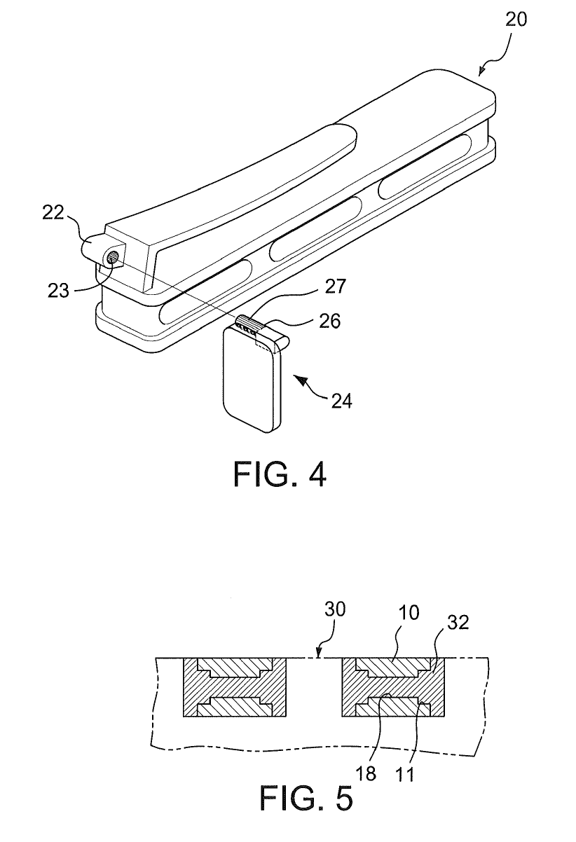

As shown in FIG. 4, the carrier body 20 in an embodiment further has a pivotal hole 22 on one end and the head 24 has a pivotal shaft 26 capable of being inserted into the pivotal hole 22 such that the head 24 is able to pivot with respect to the carrier body 20. The pivotal shaft 26 is held in the pivotal hole 22 by the frictional force therebetween and a user can swivel the head with the pivotal shaft 26 relative to the pivotal hole 22 to a desired angle relative to the carrier body 20. The outer surface of the pivotal shaft 26 and the inner surface of the pivotal hole 22 can have engaging ribs 23, 27 so as to enhance the friction force for engagement between the pivotal shaft 26 and the pivotal hole 22.

In another embodiment of the invention, the carrier body 10 is formed in a known molding process, such as injection molding. The molded carrier body 10 further has slots 18 (FIG. 1 & FIG. 5) in the recessed portion 11 and another mold 30 is provided for forming the scent portion 16 in the carrier body 10. FIG. 5 shows a method of forming the scent portion 16 in the carrier body 10. In the embodiment, several carrier bodies 10 are placed in the mold 30 and then the scent gel 32 is poured into the mold 30 and fills the slots 18 and the recessed portion 11. After the scent gel 32 is solidified, it forms the scent portion 16 and interlocks with the carrier body 10. Finally, the carrier body 10 with the scent portion 16 formed therein is removed from the mold 30. The scent gel 32 can be any kind of polymer gel containing scent material, such as polyvinylchloride (PVC), ethylene vinyl acetate (EVA), thermoplastic polyurethane (TPU) and so on.

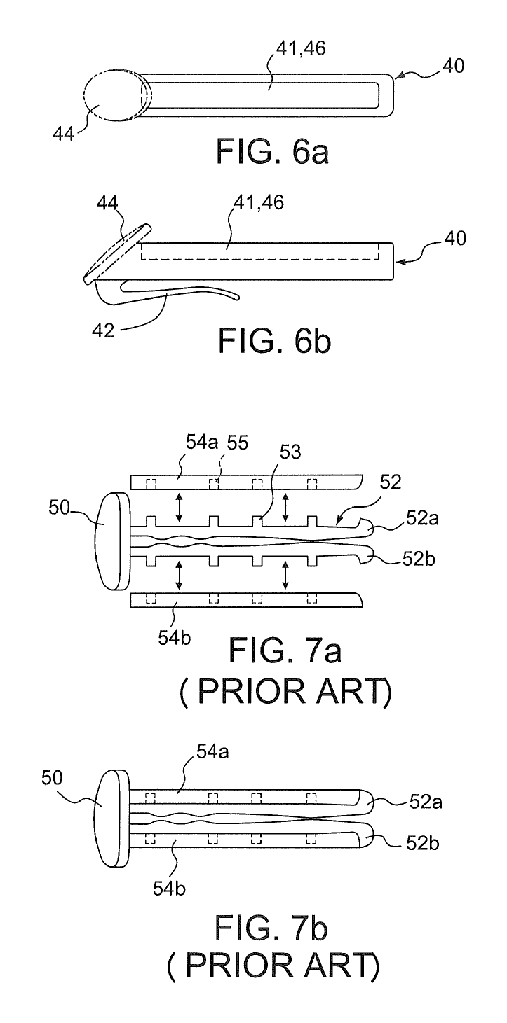

FIGS. 6a and 6b show another embodiment of the air freshener of the invention. As shown in FIGS. 6a and 6b, the air freshener of the invention includes a carrier body 40 having a recessed portion 41 thereon, a resilient clip portion 42 extending from one end of the carrier body 40 and a head 44 arranged on the end of the carrier body 40. Alternatively, the head 44 is directly formed on the end of the carrier body 40, as shown in FIG. 6. The head 44 can be omitted if it is not necessary, or alternatively be formed on the resilient clip portion 42 as described above. The head 44 can also be of various shapes as indicated previously.

In making the air freshener of the invention of the embodiment, the carrier body 40 having the above structure is formed by a known process, such as injection molding. Then, the scent gel is poured into the recessed portion 41 of the carrier body 40 and is solidified therein to form a scent portion 46. The solidification process can be carried out by chemical reaction, cooling the polymer scent gel, or adding an agent for solidification to the scent gel and activating the agent after the gel-pouring process. After the scent gel is solidified, the scent portion 46 is formed and received in the recessed portion 41 of the carrier body 40. The scent gel can be any kind of polymer gel containing scent material, such as polyvinylchloride (PVC), ethylene vinyl acetate (EVA), thermoplastic polyurethane (TPU) and so on.

As an alternative, the recessed portion 41 of the carrier body 40 can be one or a plurality of through holes so as to contain more scent gel and achieve a stronger interlock structure of the scent portion and the carrier body. In addition, the recessed portion 41 can be in various shapes to meet various demands and achieve an aesthetically desirable appearance.

Although the present invention has been described in relation to particular embodiments thereof, many other variations and modifications and other uses may become apparent to those skilled in the art. It is preferred, therefore, that the present invention be limited not by the specific descriptions herein, but only by the appended claims.

What is claimed is:

1. An air freshener, comprising:
a carrier body having a recessed portion along its periphery;
a resilient clip portion extending from the carrier body; and
a scent portion received in the recessed portion, the scent portion being at least partially in the form of an elastic band such that the scent portion is at least partially held in the recessed portion by the elasticity of the scent portion.

2. The air freshener according to claim 1, wherein a head is arranged on one end of the carrier body.

3. The air freshener according to claim 2, wherein the head is inclined relative to the carrier body.

4. The air freshener according to claim 2, wherein the carrier body has a pivotal hole on the end and the head has a pivotal shaft inserted into the pivotal hole such that the head is able to pivot with respect to the carrier body.

5. The air freshener according to claim 4, wherein the pivotal shaft is engaged with the pivotal hole by the frictional force therebetween such that the head can be pivoted and held at any pivotal angle of the head relative to the carrier body.

6. The air freshener according to claim 4, wherein the outer surface of the pivotal shaft and the inner surface of the pivotal hole each have engaging ribs to enhance the engagement between the pivotal shaft and the pivotal hole.

7. The air freshener according to claim 1, wherein the air freshener further has a head that is made of scent material and is integrally formed with the scent portion.

8. The air freshener according to claim 2, wherein the head is formed in any of the following shapes: circle, rectangle, square, doll-like shape, heart-shaped and irregular shape.

9. An air freshener, comprising:
a carrier body having a recessed portion along its periphery, the recessed portion having at least one slot;
a resilient clip portion extending from the carrier body;
a head arranged on one end of the carrier body; and
a scent gel poured and solidified in the recessed portion and the at least one slot to form a scent portion that interlocks with the carrier body.

10. The air freshener according to claim 9, wherein the head is inclined relative to the carrier body.

11. The air freshener according to claim 9, wherein the carrier body has a pivotal hole on the end and the head has a pivotal shaft inserted into the pivotal hole such that the head is able to pivot with respect to the carrier body.

12. The air freshener according to claim 11, wherein the pivotal shaft is engaged with the pivotal hole by the frictional force therebetween such that the head can be pivoted and held at any pivotal angle of the head relative to the carrier body.

13. The air freshener according to claim 11, wherein the outer surface of the pivotal shaft and the inner surface of the pivotal hole each have engaging ribs to enhance the engagement between the pivotal shaft and the pivotal hole.

14. The air freshener according to claim 9, wherein the head is made of scent material and is integrally formed with the scent portion.

15. The air freshener according to claim 9, wherein the head is formed in any of the following shapes: circle, rectangle, square, doll-like shape, heart shaped and irregular shape.

16. An air freshener, comprising:
a carrier body having a recessed portion thereon;
a resilient clip portion extending from the carrier body;
a head arranged on one end of the carrier body; and
a scent gel poured and solidified in the recessed portion.

17. The air freshener according to claim 16, wherein the head is inclined relative to the carrier body.

18. The air freshener according to claim 16, wherein the carrier body has a pivotal hole on the end and the head has a pivotal shaft capable of being inserted into the pivotal hole such that the head pivots with respect to the carrier body.

19. The air freshener according to claim 18, wherein the pivotal shaft is engaged with the pivotal hole by the frictional force therebetween such that the head can be pivoted and held at any pivotal angle of the head relative to the carrier body.

20. air freshener according to claim 18, wherein the outer surface of the pivotal shaft and the inner surface of the pivotal hole each have engaging ribs to enhance the engagement between the pivotal shaft and the pivotal hole.

21. The air freshener according to claim 16, wherein the head is formed in any of the following shapes: circle, rectangle, square, doll-like shape, heart shaped and irregular shape.

22. A method of making an air freshener, comprising the steps of:
a) forming a carrier body with a recessed portion along its periphery, at least one slot in the recessed portion and a resilient clip portion extending from one end of the carrier body;
b) arranging a head on the end of the carrier body;
c) pouring scent gel to fill the at least one slot and the recessed portion; and
d) solidifying the scent gel to form a scent portion.

23. The method of making an air freshener according to claim 22, wherein the head is formed to be inclined relative to the carrier body.

24. The method of making an air freshener according to claim 22, wherein the carrier body is formed to have a pivotal hole on the end and the head is formed to have a pivotal shaft, and the step of arranging the head includes inserting the pivotal shaft into the pivotal hole such that the head can pivot with respect to the carrier body.

25. The method of making an air freshener according to claim 24, wherein the pivotal shaft is engaged with the pivotal hole by the frictional force therebetween such that the head can pivot and hold its position at any pivotal angle of the head relative to the carrier body.

26. The method of making an air freshener according to claim 24, wherein the outer surface of the pivotal shaft and the inner surface of the pivotal hole each have engaging ribs to enhance the engagement between the pivotal shaft and the pivotal hole.

27. The method of making an air freshener according to claim 22, wherein the step of arranging the head is coincidental with the step of pouring scent gel and includes pouring scent gel in a mold to integrally form the head with the scent portion when the scent gel is solidified.

28. The method of making an air freshener according to claim 22, wherein the step of arranging the head includes integrally forming the head with the carrier body.

29. The method of making an air freshener according to claim 22, wherein the head is formed in any of the following shapes: circle, rectangle, square, doll-like shape, heart shaped and irregular shape.

30. A method of making an air freshener, comprising the steps of:
a) forming a carrier body with a recessed portion thereon and a resilient clip portion extending from one end of the carrier body;
b) arranging a head on the end of the carrier body;
c) pouring scent gel to fill the recessed portion; and
d) solidifying the scent gel to form a scent portion.

31. The method of making an air freshener according to claim 30, wherein the head is formed to be inclined relative to the carrier body.

32. The method of making an air freshener according to claim 30, wherein the carrier body is formed to have a pivotal hole on the end and the head is formed to have a pivotal shaft, and the step of arranging the head includes inserting the pivotal shaft into the pivotal hole such that the head can pivot with respect to the carrier body.

33. The method of making an air freshener according to claim 32, wherein the pivotal shaft is engaged with the pivotal hole by the frictional force therebetween such that the head can pivot and hold its position at any pivotal angle of the head relative to the carrier body.

34. The method of making an air freshener according to claim 32, wherein the outer surface of the pivotal shaft and the inner surface of the pivotal hole each have engaging ribs to enhance the engagement between the pivotal shaft and the pivotal hole.

35. The method of making an air freshener according to claim 30, wherein the step of arranging the head includes integrally forming the head with the carrier body.

36. The method of making an air freshener according to claim 30, wherein the head is formed in any of the following shapes: circle, rectangle, square, doll-like shape, heart shaped and irregular shape.

37. The air freshener according to claim 7, wherein the head is formed in any of the following shapes: circle, rectangle, square, doll-like shape, heart-shaped and irregular shape.
